(19) United States
(12) Patent Application Publication
(10) Pub. No.: US 2011/0098619 A1
(43) Pub. Date: Apr. 28, 2011

(54) SHOULDER MECHANISM FOR AN ORTHOSIS

(75) Inventors: Philippe Garrec, Gif Sur Yvette (FR); Jean-Pierre Martins, Villelaure (FR)

(73) Assignee: COMMISSARIAT A L'ENERGIE ATOMIQUE ET AUX ENERGIES ALTERNATIVES, Paris (FR)

(21) Appl. No.: 12/997,181

(22) PCT Filed: Jun. 10, 2009
(86) PCT No.: PCT/FR2009/000691

§ 371 Date: Dec. 9, 2010

(30) Foreign Application Priority Data
Jun. 10, 2008 (FR) 08 03204

(51) Int. Cl. A61F 5/00

(52) U.S. Cl. 602/20

(57) ABSTRACT

The invention relates to a shoulder mechanism for an orthosis, providing a ball type joint between a support and an orthosis upper arm, comprising a first intermediate element mounted to pivot on the support about a first pivot axis, a second intermediate element mounted to pivot on the first intermediate element about a second pivot axis, the upper arm being mounted to pivot on the second intermediate element about a third pivot axis. According to the invention, the first pivot axis extends in a longitudinal horizontal direction, the first intermediate element including a circular rail on which the second intermediate element slides and that presents a geometrical axis defining the second pivot axis that extends vertically when the upper arm is at rest, the circular rail extending to go around the shoulder on the outside.

### BACKGROUND OF THE INVENTION

In general, such an orthosis comprises an upper arm having a forearm jointed to its end. The upper arm is jointed by means of a shoulder joint to a support. The support may be stationary, or on the contrary it may be worn by the user of the orthosis. In general, it is located behind the user so as to avoid any interference during manipulation. The support is then referred to as a back.

In practice, the shoulder joint may be approximated by a ball joint, over a wide range of movement. It is known to make such a ball joint by using three pivot connections with axes that are concurrent.

For this purpose, a first intermediate element is mounted to pivot on the support about a first pivot axis. A second intermediate element is mounted to pivot on the first intermediate element about a second pivot axis perpendicular to the first and concurrent therewith. Finally, the upper arm of the orthosis is mounted to pivot on the second intermediate element about a third pivot axis perpendicular to the first and second axes and concurrent therewith. The above arrangement of the pivot axes applies when the upper arm is at rest beside the body. Whatever the position of the upper arm, the three pivot axes remain substantially concurrent.

Various embodiments are known. Starting from the support, the first pivotal connection may comprise a pivot axis that extends either vertically or horizontally. When it extends horizontally, the pivot axis may extend from back to front relative to the user in a direction that is referred to herein as longitudinal, or may it extend in a transverse direction as defined by the two shoulders of the user and perpendicular to the longitudinal direction. The directions of the axes are given herein by way of indication, and their orientations may be somewhat different, e.g. as a result of optimization.

Orthoses in which the pivot axis of the first pivotal connection extends vertically have been proposed, but they suffer from the drawback of the pivot mechanism that embodies said first pivotal connection projecting above the shoulder, and thus in the vicinity of the user's head, which is troublesome for the user, at least because this mechanism is continuously in the user's field of view. In addition, such an arrangement leads to interference with the end of the forearm when the manipulator of the orthosis bends the forearm towards the shoulder.

Orthoses in which the axis of the first pivotal connection extends in a transverse horizontal direction can be made, but the mechanism embodying this pivotal connection then projects sideways from the shoulder and is liable to interfere with the orthosis while it is being manipulated.

The invention relates more particularly to a shoulder mechanism in which the axis of the first pivotal connection extends in a longitudinal direction.

### OBJECT OF THE INVENTION

An object of the invention is to provide a shoulder mechanism for an orthosis in which the shoulder pivot mechanism is designed to minimize simultaneously any risk of singularity by the axes of the pivotal connections coming into alignment, and any risk of interference with the orthosis while it is being manipulated.

### BRIEF DESCRIPTION OF THE INVENTION

In order to achieve this object, the invention provides a shoulder mechanism for an orthosis, providing a ball type joint between a support and an orthosis upper arm, comprising a first intermediate element mounted to pivot on the support about a first pivot axis, a second intermediate element mounted to pivot on the first intermediate element about a second pivot axis, the upper arm being mounted to pivot on the second intermediate element about a third pivot axis, wherein, according to the invention, the first pivot axis extends in a longitudinal horizontal direction, the first intermediate element including a circular rail on which the second intermediate element slides and that presents a geometrical axis defining the second pivot axis that extends vertically when the upper arm is at rest, the circular rail extending to go around the shoulder on the outside.

This disposition provides several advantages. The use of a first axis that extends in a longitudinal direction makes it possible to place the actuator mechanism of the corresponding pivotal connection on the support, behind the user, such that the actuator mechanism does not risk interfering with the upper arm. The circular rail provides a pivot about a vertical axis, however it extends substantially horizontally so it does not impede the field of view of the user and it does not risk interfering with the end of the forearm when the forearm is moved towards the shoulder. Only a situation in which the upper arm is moved horizontally sideways would cause the rail to tilt about the first pivot axis from a substantially horizontal position to a substantially vertical position where it would interfere with the field of view of the user. However this situation is rare in practice. Finally, the risk of singularity by the third axis coming into alignment with the first axis is easily eliminated by providing a stroke limiter along the circular rail in order to prevent such alignment.

### BRIEF DESCRIPTION OF THE DRAWING

The invention can be better understood in the light of the figures in the accompanying drawing, in which:

FIG. 1 is a theoretical kinematic diagram showing the various pivotal connections of a shoulder mechanism of the invention, the user being represented symbolically by chain-dotted lines, in back view; and

FIG. 2 is a diagram showing one way in which the FIG. 1 shoulder mechanism can be actuated in a particular embodiment of the invention.

### DETAILED DESCRIPTION OF THE INVENTION

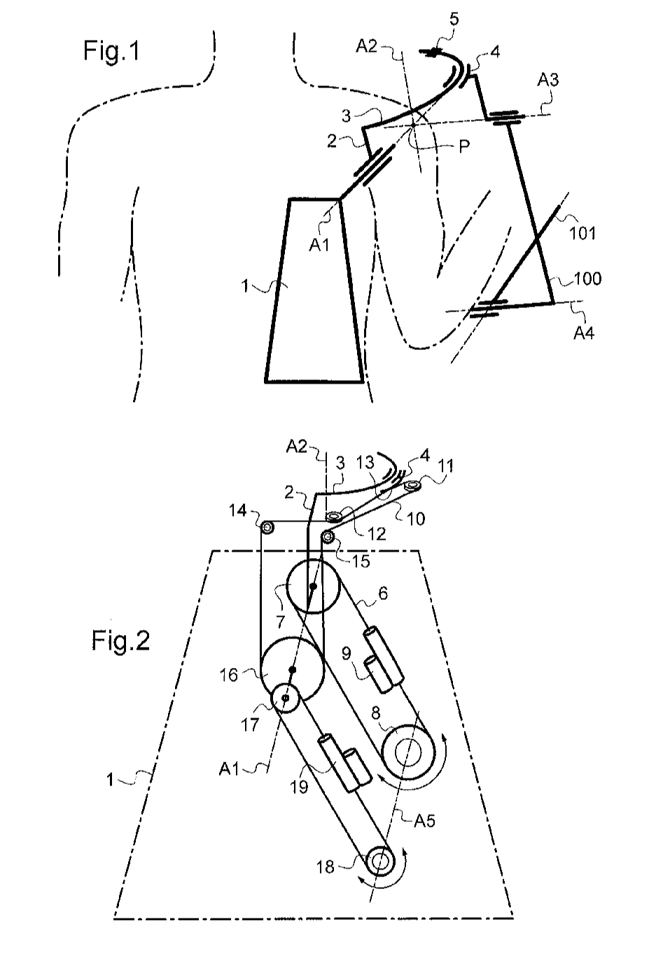

With reference to FIG. 1, the shoulder mechanism of the invention provides a ball-joint type connection between a support 1, here a back against which the user leans while the orthosis is being manipulated, and an upper arm 100. For this purpose, the shoulder mechanism of the invention comprises a first intermediate element 2 that is mounted to pivot on the support 1 by means of a pivot of axis A1 extending in a longitudinal horizontal direction (i.e., specifically, from back to front relative to the user). The first intermediate element 2 includes a circular rail 3 that extends along a circular arc centered on a geometrical axis A2 that extends vertically and that is concurrent with the axis A1. In this example, the first intermediate element 2 is shown in the position that corresponds to the upper arm being in the rest position beside the user's body. In this position, the circular rail 3 goes around the shoulder on the outside thereof and extends substantially in a horizontal plane. The field of view of the user is completely unobstructed when looking sideways towards the orthosis.

A carriage slides on the circular rail 3 of the first intermediate element, the carriage forming a second intermediate element 4 that therefore moves along a circular arc centered on the axis A2. Thus, the second intermediate element 4 is connected to the first intermediate element via a pivotal connection about the axis A2.

Finally, the upper arm 100 is pivotally mounted on the second intermediate element 4 via a pivot of axis A3 extending in a transverse direction. The axis A3 is perpendicular to the axes A1 and A2 and it is concurrent with both of them at point P in the figure that coincides substantially with the operator's shoulder.

The three pivotal connections arranged in this way, of axes that are always concurrent regardless of the position of the upper arm, together form the ball-joint connection between the support 1 and the upper arm 100.

An end-of-stroke abutment 5 on the circular rail 3 prevents the second intermediate element 4 from reaching a position in which the axis A3 comes into alignment with the axis A1, thus ensuring in very simple manner that there is no alignment singularity.

The upper arm 100 carries a forearm 101 that is jointed to the upper arm 100 about an axis A4 parallel to the axis A3.

According to a particular aspect of the invention, shown in FIG. 2, pivoting of the first intermediate element 2 about the axis A1 is controlled by means of a cable transmission 6 that extends between a first pulley 7 coupled to the first intermediate element 2 and a second pulley 8 on a parallel axis A5 that is pivotally mounted on the support 1. An actuator 9 acts on the cable to cause it to move forwards or backwards, thereby causing the first pulley 7 to turn, which in turn causes the first intermediate element to pivot, thereby driving pivoting of the upper arm about the axis A1.

According to another particular aspect of the invention, the sliding of the second intermediate element 4 along the circular rail 3 is likewise controlled by a cable transmission 10. More precisely, pulleys 11 and 12 pivotally mounted on the first intermediate element 2 cause the cable 10 to pass in the proximity of the circular rail 3. The second intermediate element 4 is attached to the cable 10 at a point 13. Then pulleys 14 and 15, likewise pivotally mounted on the first intermediate element 2, serve to deflect the cable 10 towards a drive pulley 16 pivotally mounted on the support 1 about the axis A1. The drive pulley 16 is in turn driven by a cable assembly (comprising pulleys 17 and 18 and an actuator 19) entirely similar to that used for controlling pivoting of the first intermediate element 2 about the axis A1, in which the pulley 17 is coupled to the drive pulley 16 in order to cause it to turn, and thus cause the cable 10 to move, thereby causing the second intermediate element 4 to slide along the circular rail 3. The pulleys 7 and 17 are on a common axis, as are the pulleys 8 and 18.

The actuator assembly made up of the pulleys 7, 8, the actuator 9, and the associated cable, and the actuator assembly made up of the pulleys 17, 18, of the actuator 19, and of the associated cable are both mounted on the support 1 behind the user so that the user's field of view is not obstructed, while nevertheless any risk of interference with the upper arm is avoided.

As for the assembly made up of the cable 10 and the pulleys 11, 12, 14, and 15, it is secured to the first intermediate element 2 and therefore travels with it as it pivots about the axis A1. To avoid any unwanted sliding of the second intermediate element 4 along the circular rail 3 during pivoting of the first intermediate element 2 about the axis A1, it is appropriate to organize equivalent pivoting of the drive pulley 16 by means of the corresponding actuator assembly.

In FIG. 2, the shafts, connections, and other supports that serve to connect the pulleys 11, 12, 14, and 15 with the first intermediate element 2, and the shaft, connections, and other supports that enable the pulleys 7, 8, 17, and 18 and the actuators 9 and 19 to be secured to the support 1 are omitted for greater clarity.

In this example, the axes A1, A2, and A3 are concurrent, such that the connection defined in this way is a perfect ball joint (ignoring uncertainties of positioning and deformation). Nevertheless, it is also possible to devise a shoulder mechanism for an orthosis of the invention in which the axes are not exactly concurrent.

In reality, human shoulder joints do not correspond to perfect ball joints. In practice, for a shoulder joint that is to be fitted with an orthosis, it is possible to define a dispersion sphere that is the smallest sphere in which an arbitrary point of the upper arm of the person fitted with the orthosis moves during three-dimensional movements of the arm. This sphere may be determined by experimental procedures, in particular by measuring movements. This sphere is characterized in particular by a radius, referred to herein as Rₕᵤₘₑᵣᵤₛ and it is centered on a point close to the theoretical center of the perfect ball joint that represents the joint in question as well as possible. The radius Rₕᵤₘₑᵣᵤₛ of the dispersion sphere of a human shoulder is typically of the order of a few centimeters.

It is then advantageous to define a shoulder orthosis mechanism of the invention having a dispersion sphere of center that coincides with the center of the dispersion sphere of the joint to be fitted with the orthosis, and of radius Rₒᵣₜₕₒₛᵢₛ that is no greater than the radius of the dispersion sphere of the arm Rₕᵤₘₑᵣᵤₛ. In this example, the radius Rₒᵣₜₕₒₛᵢₛ of the dispersion sphere of the shoulder mechanism of the invention, also referred to as concurrency error, is by definition the sum of the distance between the axis A1 and the axis A2 plus the distance between the axis A2 and the axis A3. Each of the distances mentioned corresponds to the lengths of a segment perpendicular to the two axes in question.

If the radius Rₕᵤₘₑᵣᵤₛ of the dispersion sphere of the shoulder to be fitted with the prosthesis is equal to 40 millimeters (mm), then a concurrency error of no more than 40 mm is used, and it is possible to design a shoulder orthosis mechanism of the invention having axes that are, for example, spaced apart in pairs by a distance that is equal to 20 mm. Naturally, a mechanism as shown in FIG. 1 has axes that are concurrent, such that the concurrency error is zero.

Preferably, the shoulder orthosis mechanism is provided with adjustment means to cause the center of the dispersion sphere of the fitted shoulder to coincide with the center of the dispersion sphere of the orthosis as designed in this way.

In order to cover both of these configurations (concurrent axes or non-concurrent axes), the more general term used below is that of a ball-joint type connection.

The invention is naturally not limited to the above description, but covers any variant coming within the ambit defined by the claims.

In particular, although cable transmissions are preferred since they are generally more compact, more reliable, and present excellent regularity of transmitted force, it is possible to use other methods of actuation. Nevertheless, it remains advantageous to provide for a return of the movement of the second intermediate element towards the support by means of a cable as described herein or by means of a linkage or by means of shafts so that the corresponding actuator can remain stationary and be located on the support 1.

1. A shoulder mechanism for an orthosis, providing a ball type joint between a support and an orthosis upper arm, comprising a first intermediate element mounted to pivot on the support about a first pivot axis, a second intermediate element mounted to pivot on the first intermediate element about a second pivot axis, the upper arm being mounted to pivot on the second intermediate element about a third pivot axis, wherein the first pivot axis extends in a longitudinal horizontal direction, the first intermediate element including a circular rail on which the second intermediate element slides and that presents a geometrical axis defining the second pivot axis that extends vertically when the upper arm is at rest, the circular rail extending to go around the shoulder on the outside.

2. A shoulder mechanism according to claim 1, wherein the movement of the second intermediate element along the circular rail of the first intermediate element is controlled by an actuator placed on the support.

3. A shoulder mechanism according to claim 2, wherein the second intermediate element is coupled to a cable that is associated with pulleys that deflect the cable towards a drive pulley centered on the pivot axis of the first intermediate element on the support, the drive pulley being driven by the actuator placed on the support.

4. A shoulder mechanism according to claim 3, wherein the actuator is a cable actuator that acts on a cable mounted between two pulleys of parallel axes, one of them lying on the same axis as the drive pulley and being coupled thereto.

5. A shoulder mechanism according to claim 1, wherein the pivoting of the first intermediate element on the support is controlled by a cable actuator that acts on a cable mounted between two axes of parallel axes, one of which coincides with the axis A1, and the corresponding pulley being coupled to the first intermediate element.

6. A shoulder mechanism according to claim 1, wherein the first, second, and third pivot axes are substantially concurrent.

7. A shoulder mechanism according to claim 1, wherein the sum of a distance between the first and second pivot axes plus a distance between the second and third pivot axes is selected to be less than a radius of a dispersion sphere of a shoulder fitted with the shoulder mechanism.
